(19) United States
(12) Patent Application Publication
(10) Pub. No.: US 2011/0208011 A1
(43) Pub. Date: Aug. 25, 2011

(54) SYSTEM AND METHOD FOR GUIDING OF GASTROINTESTINAL DEVICE THROUGH THE GASTROINTESTINAL TRACT

(76) Inventor: Shomron Silan Ben-Horin, Ramat Efal (IL)

(21) Appl. No.: 12/738,641

(22) PCT Filed: Oct. 7, 2008
(86) PCT No.: PCT/IL08/01337

§ 371 Date: Oct. 19, 2010

Related U.S. Application Data
(60) Provisional application No. 60/980,474, filed on Oct. 17, 2007.
(60) Provisional application No. 61/057,890, filed on Jun. 2, 2008.

(51) Int. Cl. A61B 19/00

(52) U.S. Cl. 600/300

(57) ABSTRACT

Systems and methods for guidance of a gastrointestinal device through a gastrointestinal tract are provided. A gastrointestinal guidewire is positioned through the gastrointestinal tract, by introduction of an introducing element into the gastrointestinal tract. One end of the gastrointestinal guidewire is attached to the introducing element, and the other end of the gastrointestinal guidewire is attached to an anchoring element, anchored to a location outside of the gastrointestinal tract. Movement of the introducing element through the gastrointestinal tract results in positioning of the guidewire through the gastrointestinal tract. The guidewire is then used as a scaffold to guide the gastrointestinal device, wherein the gastrointestinal device may be externally controlled. This allows for speeding up, slowing down, reversal and stopping of the gastrointestinal device during its descent through the gastrointestinal tract.

### FIELD OF THE INVENTION

The present invention relates to systems and methods for guiding of a gastrointestinal device through the gastrointestinal tract. A gastrointestinal guidewire is positioned through the gastrointestinal tract and is configured to act as a scaffold for guiding the gastrointestinal device.

### BACKGROUND OF THE INVENTION

Access to the lumen of the gastrointestinal tract is useful for diagnosing and treating diverse disorders such as inflammation, cancer and gastrointestinal bleeding. Imaging of the lumen can be accomplished by various flexible fiberoptic endoscopes, introduced through the mouth (gastroscopes or enteroscopes) or the anus (colonoscopes). These procedures, especially colonoscopy and enteroscopy, require various manual maneuvers by the operator, usually a physician, in order to advance the endoscope while avoiding its looping, thereby making insertion of the endoscope technically demanding. Moreover, the patient discomfort that accompanies these maneuvers and the looping of the endoscope necessitates sedation of most patients undergoing these procedures. In addition, diagnostic and therapeutic endoscopic access to the small intestine beyond the duodenum is limited because of its long tortuous course, and because of its redundancy. These topographical and mechanical features of the small intestine hamper the advancement of endoscopes into the small intestine by the usual maneuvers of forward pushing of the endoscope alternating with occasional retraction in order to reduce loops formed by the endoscope tube. Recently, a double-balloon enteroscope (DBE) has been introduced into the field, for the purpose of endoscopic studies of the small intestine. The DBE consists of two balloons which are inflated and deflated sequentially by the operator, in order to alternately fix and advance an overtube and an internal enteroscope through the intestine. However, this is a technically demanding and lengthy procedure, necessitating heavy sedation of the patient, and is also seldom successful in traversing the entire small intestine in a single procedure from a single orifice (i.e. mouth or anus).

Due to these obstacles, capsule endoscopy is currently the mainstay method for diagnosis of disorders of the small intestine, and is also gaining acceptance for visualizing the upper and lower gastrointestinal tract. This method comprises oral administration to a subject of a capsule equipped with a camera that transmits the images obtained to an external recorder. The images are viewed on a screen by an operator at a later time. One such capsule is disclosed in U.S. Pat. No. 5,604,531. A major limitation of capsule endoscopy is that operator-controlled movement is unavailable at present, and images are obtained while the capsule advances passively along the gastrointestinal tract solely by the force of intestinal motility. This, in turn, hampers visualization of particular segments of interest, or of the complete length of the tract, as movement of the capsule is random. Indeed, some capsules do not traverse the entire small intestine before their battery runs out, and similarly, sections of interest may be overlooked or missed because of rapid peristalsis carrying the capsule briskly forward across a lesion. The random and uncontrollable capsule movement also precludes operator-guided lesion-specific intervention, such as the procurement of tissue for biopsy, manipulations to control bleeding or targeted drug delivery. Moreover, the period during which the capsule traverses the gastrointestinal tract generally takes place outside of the clinic (at home, for example), during which time a film of the gastrointestinal tract is produced. Because the patient is outside of the clinic, the possibility of biopsy is eliminated. Moreover, the produced film is generally lengthy (approximately 7 hours), necessitating long hours of review on the part of the doctor.

The passive peristalsis-dependent movement of capsules is also a major obstacle in the use of capsule endoscopy for colon visualization, as physiologic colonic transit time is in the order of 48 hours i.e. much longer than small intestine transit time. While the colon transit time can be shortened by vigorous purgative and pro-kinetic regimens, such procedures are disagreeable for patients and can entail a lack of patient compliance. Moreover, even then a significant fraction of capsules still do not traverse the entire colon and are not expelled from the anus before their battery runs out at the end of study, thereby compromising and adversely affecting the diagnostic utility of capsules for studies of the human colon.

U.S. Pat. No. 7,226,410 discloses a device for performing medical procedures inside a body lumen such as the gastrointestinal tract, wherein the device comprises a capsule attached to a cable, wherein the distal end of the cable is arranged in a loop which unfurls within the gastrointestinal lumen and lays down a track upon which the capsule is manipulated forward or backward by a propulsion mechanism. According to the disclosure, both the laying down of the cable and the movement of the capsule require manipulations by an operator i.e. the cable is laid out as the operator slides it through the device, and the capsule is propelled along the cable as the operator activates a motion control unit. Other string capsules such as the Watson-Crosby capsule and the Given Imaging string capsule are configured to be ingested with a string or tube attached thereto, and they are retracted back from the oral cavity and therefore are limited to investigation of the upper part of the small intestine.

U.S. Pat. No. 6,632,171 discloses a clamp device for manually positioning an endoscopic capsule within the gastrointestinal tract. U.S. Pat. No. 6,884,213 discloses a hydraulically operated injecting device for positioning an endoscopic capsule within the gastrointestinal tract. U.S. Pat. No. 6,936,003 discloses a device which includes one or more extendible arms, termed proboscises, and an in vivo medical instrument, such as an endoscopic capsule, wherein the proboscises can perform different functions, including moving and propelling the device. U.S. Pat. No. 6,986,738 discloses a system comprising a tube and an in vivo sensing device that is changeably connected to the tube, for example, through an elastic cord or wire, and wherein the position of the sensing device with respect to the tube can be changed. U.S. Pat. No. 6,939,290 discloses an in vivo sensing device comprising a sensor, such as an endoscopic capsule, and a non-protruding magnetohydrodynamic propulsion system. All of the aforementioned positioning systems are considerably dependent on a high level of technical skill of the operator.

Digestible diagnostic capsules with extractable strings (often termed gastric strings) are used in the stomach for measurement or diagnosis. A patient holds the free end of the gastric string, and then swallows the capsule, during which time the string exits the capsule as it travels through the esophagus and enters the stomach. The capsule then either dissolves or passes through the patient's digestive system, leaving the string within the stomach. After a certain period of time the string is withdrawn, and the end of the string that was in the stomach is subsequently tested for various indicators, such as the presence of certain microorganisms or to measure the pH of the stomach content. Such devices are disclosed, for example, in U.S. Pat. Nos. 3,528,429; 3,683,890, and 5,738,110. These devices, however, are not designed to traverse the lower gastrointestinal tract such as the small and large intestines.

Ramirez et al. disclose a wireless capsule endoscopy device attached to a string to allow its controlled movement up and down the esophagus. After ingestion, the capsule obtains images of the esophagus and stomach, and is then withdrawn through the mouth by the operator for repeated future use (Ramirez FC et al, Gastrointestinal Endoscopy 2005; 61(6) 741-6).

U.S. Pat. No. 7,037,275 discloses a gastrointestinal sampling device which is a capsule comprising a malleable drag material, inter alia absorbent string, cotton, sampling cloth, wool, acrylic, nylon, plastic, chain links or finely woven metal, and a protective sheath which is deployed around the drag material upon withdrawal of the device. According to the disclosure, a weight is attached to one end of the drag material in order to manipulate its position by application of an external magnetic force.

The inability to control the movement of endoscopes, endoscopic capsules, as well as other diagnostic capsules (e.g. pH recording capsules, chemical sampling capsules, etc) or gastrointestinal devices significantly diminishes their diagnostic and therapeutic utility. Additionally, the tortuous and redundant nature of the small intestine poses a significant obstacle for manual advancement of endoscopes through the small intestine. Accordingly, there is a great need in the art for a means of controlling the movement of diagnostic and therapeutic gastrointestinal devices.

U.S. Pat. No. 5,879,325 discloses introduction of a tube through a gastrointestinal tract for the purpose of introducing substances at locations along the gastrointestinal tract. However, the tube is pulled through the gastrointestinal system from a location external to the gastrointestinal system, which may cause discomfort and limits the options for introduction of the tube.

There is thus a need for a system and method for guidance of gastrointestinal devices throughout the gastrointestinal tract, and particularly through the small and large intestines.

### SUMMARY OF THE INVENTION

There is thus provided, in accordance with embodiments of the present invention, a system for guidance of a gastrointestinal device through a gastrointestinal tract. The system includes an introducing element having an introducing element distal end, an introducing element proximal end, an exterior portion and an interior portion, a guidewire attachment point at the introducing element, an anchoring element external to the introducing element, and a gastrointestinal guidewire having a guidewire proximal end, a guidewire distal end, and a guidewire body extending from the guidewire proximal end to the guidewire distal end. The guidewire body has a length sufficient to be positioned through at least to the beginning of the colon of the gastrointestinal tract. The guidewire proximal end is attached to the anchoring element and in some embodiments, the guidewire distal end is positioned within the interior portion of the introducing element and is attached to the guidewire attachment point. At least a portion of the guidewire body is configured to be released from the interior portion into the gastrointestinal tract as the introducing element moves through the gastrointestinal tract. The gastrointestinal guidewire is then configured to be used as a scaffold for guiding a gastrointestinal device through the gastrointestinal tract.

In some embodiments, the introducing element is a swallowable element, such as a swallowable capsule. The distal end of the introducing element may be lighter and/or narrower than the proximal end of the introducing element. The introducing element may be digestible, biodegradable or dissolvable, or a combination thereof. Alternatively, the introducing element may be indigestible, and excreted in substantially intact form from the body. The introducing element may further include an indicator that is detectable by an external detection method, such as, for example, one or multiple radiopaque markers, RFID tag, or metal component detectable by a magnetometer.

In accordance with some embodiments of the present invention, the attachment point is at an interior wall of said introducing element and said introducing element further comprises a guidewire outlet at said introducing element proximal end. In accordance with additional embodiments, the guidewire outlet may further include a one-way valve. Alternatively, the guidewire attachement point may be at an exterior wall of the introducing element.

In some embodiments, a rotatable spool is positioned within the introducing element, and the guidewire attachment point is on the rotatable spool. In other embodiments, the guidewire attachment point is at an interior wall of the introducing element. The introducing element may be spherical, hemispherical, tapered, conical, cylindrical or ovoid or any other suitable shape.

In accordance with some embodiments of the present invention, the introducing element comprises a plurality of elements each having a separate shape, such as, for example, spherical, hemispherical, tapered, conical, cylindrical, cuboid, a wing, an arm and ovoid.

In accordance with some embodiments of the present invention, the gastrointestinal guidewire may comprise at least one of: a biodegradable material, a non-biodegradable material, a radiopaque material, a hollow tube, and a hollow tube with a slit, or a combination thereof.

The gastrointestinal device may be a diagnostic capsule modified to be externally manipulated along the gastrointestinal guidewire. Alternatively or additionally, the gastrointestinal device may comprise one or more of a comprises one or more of an imaging device, an endoscope, a biopsy device, or a luminal content sampling device, electrocautery device, drug delivery device, surgical device or instrument, or a combination thereof.

The gastrointestinal device may further include a magnet or a motor or an externally controllable wire.

In accordance with some embodiments of the present invention, there is provided a system for guidance of a gastrointestinal device through a gastrointestinal tract. The system comprises an introducing element having an introducing element distal end, an introducing element proximal end, an exterior portion and an interior portion. A guidewire outlet is provided at the proximal end of the introducing element, a guidewire attachment point is provided in the interior portion of the introducing element, and an anchoring element is provided external to the introducing element. The system comprises a gastrointestinal guidewire having a guidewire proximal end, a guidewire distal end, and a guidewire body extending from the guidewire proximal end to the guidewire distal end, the guidewire body having a length sufficient to be positioned through an entire length of the gastrointestinal tract. The guidewire proximal end is attached to the anchoring element. The guidewire distal end is positioned within the interior portion and attached to the guidewire attachment point, Att least a portion of the guidewire body is configured to be released from the interior portion through the guidewire outlet into the gastrointestinal tract as the introducing element traverses at least a portion of the gastrointestinal tract.

In accordance with some embodiments of the present invention, there is provided a system for guidance of a gastrointestinal device through a gastrointestinal tract, the system comprising an introducing element having an introducing element distal end, an introducing element proximal end, an exterior portion and an interior portion; a guidewire outlet at the proximal end of the ntroducing element; a guidewire attachment point in the interior portion of the introducing element; and an anchoring element external to the introducing element. The system further comprises a gastrointestinal guidewire having a guidewire proximal end, a guidewire distal end, and a guidewire body extending from the guidewire proximal end to the guidewire distal end, the guidewire body having a length sufficient to be positioned through an entire length of the gastrointestinal tract. The guidewire proximal end is attached to the anchoring element, the guidewire distal end is positioned within the interior portion and attached to the guidewire attachment point. At least a portion of the guidewire body is configured to be released from the interior portion through the guidewire outlet into the gastrointestinal tract as the introducing element traverses at least a portion of the gastrointestinal tract. The gastrointestinal guidewire is configured to be used as a scaffold for guiding a gastrointestinal device through the gastrointestinal tract.

In accordance with some embodiments of the present invention, there is provided a system for guidance of a gastrointestinal device through a gastrointestinal tract, the system comprising an introducing element having an introducing element distal end, an introducing element proximal end, an exterior portion and an interior portion; a guidewire attachment point at the introducing element; and an anchoring element external to the introducing element. The system further comprises a gastrointestinal guidewire having a guidewire proximal end, a guidewire distal end, and a guidewire body extending from the guidewire proximal end to the guidewire distal end, the guidewire body having a length sufficient to be positioned through an entire length of the gastrointestinal tract. The guidewire proximal end is attached to the anchoring element, the guidewire distal end is attached to the guidewire attachment point, and at least a portion of the guidewire body is configured to be pulled from the anchoring element into the gastrointestinal tract as the introducing element traverses at least a portion of the gastrointestinal tract. The gastrointestinal guidewire configured to be used as a scaffold for guiding a gastrointestinal device through the gastrointestinal tract.

In accordance with some embodiments of the present invention, there is provided a system for guidance of a gastrointestinal device through a gastrointestinal tract, the system comprising an introducing element having an introducing element distal end, an introducing element proximal end, an exterior portion and an interior portion; a guidewire outlet at the proximal end of the introducing element; and an anchoring element external to the introducing element. The system further comprises a gastrointestinal guidewire having a guidewire proximal end, a guidewire distal end, and a guidewire body extending from the guidewire proximal end to the guidewire distal end, the guidewire body having a length sufficient to be positioned through an entire length of the gastrointestinal tract, the guidewire proximal end attached to the anchoring element, the guidewire distal end positioned within the interior portion, and at least a portion of the guidewire body configured to be released from the interior portion through the guidewire outlet into the gastrointestinal tract as the introducing element traverses at least a portion of the gastrointestinal tract. The gastrointestinal guidewire is configured to be used as a scaffold for guiding a gastrointestinal device through the gastrointestinal tract.

In accordance with additional embodiments of the present invention, there is provided a method for controlling a gastrointestinal device within the gastrointestinal tract of an individual. The method includes providing an introducing element configured to advance through the gastrointestinal tract, an anchoring element, and a guidewire attached to the introducing element and the anchoring element. The method further includes anchoring the anchoring element to a location outside of the gastrointestinal tract or in the subject's mouth, introducing the introducing element into the gastrointestinal tract and allowing the introducing element to traverse (by force of natural peristalsis, for example) at least a portion of the gastrointestinal tract, wherein during the traversal, the guidewire is positioned through the traversed portion of the gastrointestinal tract, and thereafter guiding a gastrointestinal device through the gastrointestinal tract via the guidewire, wherein the guiding includes externally controlling movements of the gastrointestinal device.

In some embodiments of the present invention, the guidewire may be initially at least partially positioned within the introducing element, and during the traversal by the introducing element, the guidewire is configured to be released from within the introducing element. In some embodiments, the anchoring is done by attaching to an oral cavity, a nasal cavity, or a location outside of a body of the individual. Introduction of the introducing element may be done by swallowing or by other means, such as by use of an endoscope. In some embodiments, the introducing element is allowed to traverse the entire gastrointestinal tract. The introducing element may be removed by removing or expelling it from the anus, or from another point along the gastrointestinal tract via an endoscope. In some embodiments, the introducing element is degraded within the body of the subject. Once the gastrointestinal guidewire is in place, the gastrointestinal device may then be guided, for example, by either remotely controlling a remotely controllable motor attached to the gastrointestinal device or by controlling a second wire attached to the gastrointestinal device. The gastrointestinal device may be sped up, slowed down, reversed, or stopped, or an angle of the gastrointestinal device may be adjusted with regard to thelongitudinal axis of the gastrointestinal tract, or other wise controlled.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1A is a schematic cross-sectional illustration of a system for positioning of a gastrointestinal guidewire through a gastrointestinal tract, in accordance with embodiments of the present invention;

FIG. 1B is a schematic cross-sectional illustration of a system for positioning of a gastrointestinal guidewire through a gastrointestinal tract, in accordance with additional embodiments of the present invention;

FIG. 2 is a schematic cross-sectional illustration of a system for positioning of a gastrointestinal guidewire through a gastrointestinal tract, in accordance with yet additional embodiments of the present invention;

FIG. 3 is an illustration of a gastrointestinal guidewire positioned throughout a gastrointestinal tract, in accordance with embodiments of the present invention;

FIG. 4 is an illustration of a system for positioning of a gastrointestinal guidewire through a gastrointestinal tract, in accordance with additional embodiments of the present invention;

FIGS. 5A-5C are illustrations of a gastrointestinal guidewire having a hollow tube configuration, in accordance with embodiments of the present invention;

FIG. 5D is an illustration of wheeled gastrointestinal device for movement along guidewire in accordance with embodiments of the present invention;

FIGS. 6A-6C are illustrations of steps of a method of positioning a gastrointestinal guidewire in the gastrointestinal tract, in accordance with embodiments of the present invention;

FIG. 7 is a flow-chart illustration of the method of FIGS. 6A-6C; FIG. 8 is an illustration of an introducing element with wings, in accordance with embodiments of the present invention;

FIG. 9 shows two views of an exemplary introducing element and guidewire according to the present invention; and

FIG. 10 shows photographs depicting the results of an illustrative experiment in a pig as the animal model. FIG. 10A shows the wire extending from within the anus of the pig and held with the introducing element in the hand of an investigator; after expulsion of introducing element from the anus of the pig; FIG. 10B shows the attachment of the wire to the abdominal wall and thereafter to the stomach wall; FIG. 100 shows the anchored wire extending along the stomach; FIG. 10D shows the wire passing from the stomach to the duodenum; FIG. 10E shows the wire passing along the small bowel; FIG. 10F shows the wire passing from the ileum to the cecum; and FIG. 10G shows the wire passing along the large bowel.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention is directed to systems and methods for controllable guidance of a gastrointestinal device through a gastrointestinal tract. The principles and operation of systems and methods according to the present invention may be better understood with reference to the drawings and accompanying descriptions.

Before explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

### Definitions

As used herein “gastrointestinal guidewire” refers to a continuous length of cable, wire, string, thread, filament, tubing or other material or construction which may be positioned along a length of the gastrointestinal tract, which is appropriate for mounting thereupon or inserting therein, of a gastrointestinal device. The length of the gastrointestinal tract traversed by the gastrointestinal guidewire is preferably the entire length of the gastrointestinal tract between the oral cavity and the anus, but it may also be a portion thereof. According to invention, the gastrointestinal guidewire is positioned in a configuration which substantially follows the route of the portion of the gastrointestinal tract which it traverses.

As used herein “gastrointestinal peristalsis” refers to one or more types of coordinated or uncoordinated contractions that normally function to propel food and waste through the various organs of the gastrointestinal system, including the oral cavity, throat, esophagus, stomach, small intestine, large intestine and rectum. Accordingly, gastrointestinal peristalsis includes one or more of swallow-induced peristalsis (also termed primary peristalsis); esophageal peristalsis (also termed secondary peristalsis); gastric peristalsis (primary, secondary or tertiary), and intestinal peristalsis (primary, secondary or tertiary).

As used herein “introducing element” refers to an object which is configured to fit through a gastrointestinal tract and to traverse at least a portion of the gastrointestinal tract in response to peristaltic contractions.

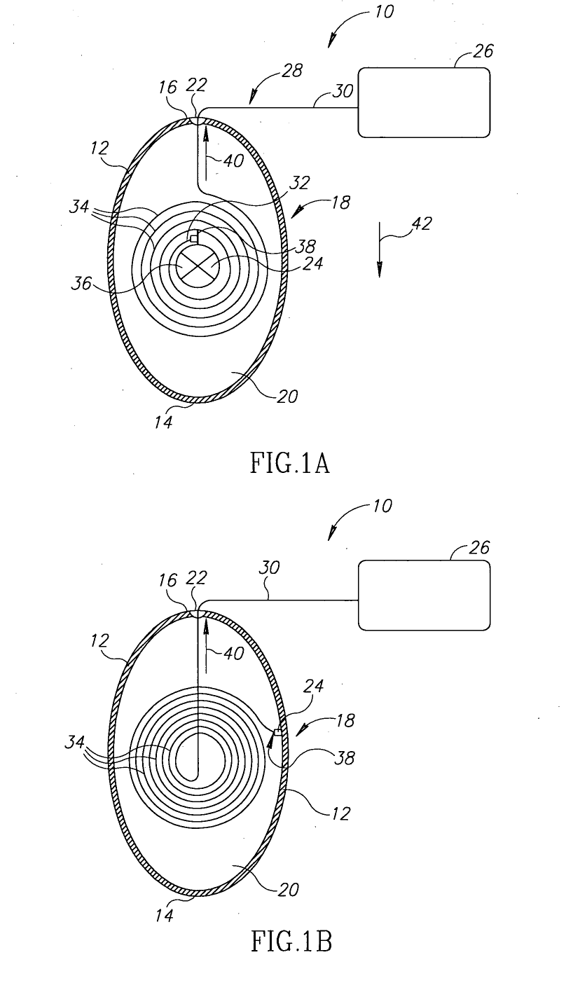

Reference is now made to FIG. 1A, which is a schematic illustration of a system 10 for positioning of a gastrointestinal guidewire through a gastrointestinal tract, in accordance with embodiments of the present invention. System 10 includes an introducing element 12 having an introducing element distal end 14 and an introducing element proximal end 16. Introducing element 12 includes an exterior portion 18, which, upon introduction of introducing element 12 into the gastrointestinal tract (for example, by swallowing or by use of an endoscope), is exposed to the gastrointestinal tract. Introducing element 12 further includes an interior portion 20. In some embodiments, interior portion 20 is hollow. In some embodiments, a guidewire outlet 22 is positioned at introducing element proximal end 16. Guidewire outlet 22 is an opening of sufficient diameter to allow a guidewire to slide therethrough. Interior portion 20 of introducing element 12 includes a guidewire retainer 24 for at least partially retaining a gastrointestinal guidewire 28 in interior portion 20, as will be explained hereinbelow.

Introducing element 12 and more particularly exterior portion 18 of introducing element 12 may be made of any suitable inert material or combination of materials, for example metal encased in a plastic coating. Introducing element 12 may be configured in any of a variety of shapes, or may comprise a plurality of elements each having a separate shape, including for example, spherical, hemispherical, tapered, conical, cylindrical and ovoid, and may be, for example, a swallowable element, such as a swallowable capsule. Introducing element 12 may be of any suitable size, and in some embodiments may be a capsule which is of a swallowable size ranging up to 36×18 mm. For example, swallowable capsules having dimensions of 1×3 cm are currently used for diagnostic procedures. The introducing element 12 of the present application may be similar in size and structure to such swallowable capsules, but is not limited to such designs.

In some embodiments, introducing element distal end 14 may be configured to weigh less than introducing element proximal end 16 so that introducing element 12 remains substantially oriented during its descent. In some embodiments, introducing element distal end 14 may be narrower than introducing element proximal end 16, so that once introducing element exits the small intestines and enters the large intestines, backward movement of introducing element 12 into the small intestines may be avoided. In this way, if introducing element 12 remains in the large intestine, gastrointestinal guidewire 28 will minimally be positioned along the small intestine, allowing for subsequent introduction of medical devices to the small intestine. In some embodiments, introducing element 12 may further include a sensor for sensing pressure, pH, peristaltic contractions, lumen diameter or other physiological parameters which may indicate when introducing element 12 moves from one area of the gastrointestinal tract to another. Additionally, as shown in FIG. 8, an expanding portion 17 may be included on introducing element proximal end 16, wherein the expanding portion can be automatically expanded once the sensor senses that the small intestines have been traversed. Expanding element 17 may, for example, be comprised of angled wings attached at an angle to introducing element 12, wherein the wings may extend outwardly at proximal end 16, thereby mechanically preventing introducing element 12 from being dragged back into the small intestine. Expanding element 17 may be comprised of any suitable material.

In some embodiments, introducing element 12 is digestible, dissolvable, or biodegradable, or a combination thereof. For example, introducing element 12 may be comprised of dissolvable gelatin. Use of a digestible introducing element 12 can be advantageous for facilitating patient compliance. In yet additional embodiments, introducing element 12 is a non-dissolvable introducing element, or alternatively comprises a dissolvable material coated with a non-dissolvable material. For example, introducing element 12 may be comprised of gelatin coated with a non-toxic material that readily passes through the digestive tract, such as enamel, porcelain, a polymer (such as a polycarbonate polymer, a polysaccharide polymer or a combination thereof), synthetic rubber or latex rubber. The coating may be carried out either by dipping or brush coating. In some embodiments, introducing element 12 is comprised of an elastic or semi-elastic material. In some embodiments, introducing element 12 incorporates an image detection means. In some embodiments, introducing element 12 is marked with an indicator that is detectable by an external detection method, such as, for example, one or multiple radiopaque markers, RFID tag, or metal component detectable by a magnetometer, or a combination thereof. In some embodiments, introducing element 12 may be comprised of an electrically conductive material (with an insulating layer thereon) so as to enable it to conduct electricity for the purpose of augmenting the movement of introducing element or the gastrointestinal device, for example.

System 10 further includes an anchoring element 26 exterior to introducing element 12. The anchoring element may be of various configurations, including but not restricted to, an adhesive tape attached to the subject's face, a band strapped and fastened around a part of the subject's head or neck, or a mold attached to the subject's palate or teeth. Gastrointestinal guidewire 28 includes a guidewire proximal end 30 attached to anchoring element 26, and a guidewire distal end 32 attached to a guidewire retainer 24 within introducing element 12 (or to introducing element 12 itself, as will be described with respect to FIG. 1B) at a distal guidewire attachment point 38.

Attachment point 38 may be at an interior wall of introducing element 12. Alternatively, attachment point 38 may be on an exterior wall of introducing element 12.

Attachment point 38 may include a pin, a clip, an adhesive, or any other suitable means of attachment, or may be integrally formed with introducing element 12. In some embodiments, attachment point 38 is configured such that introducing element 12 may be easily detached from gastrointestinal guidewire 28 after introducing element 12 is retrieved from the anus at the end of its descent through the gastrointestinal tract.

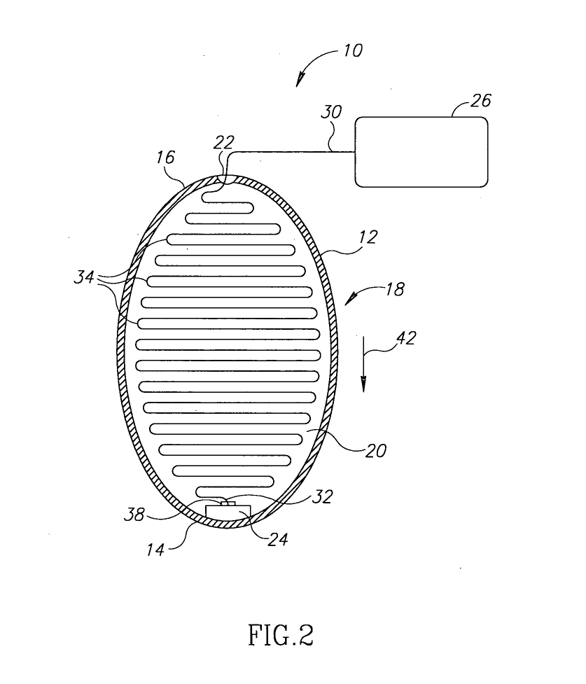

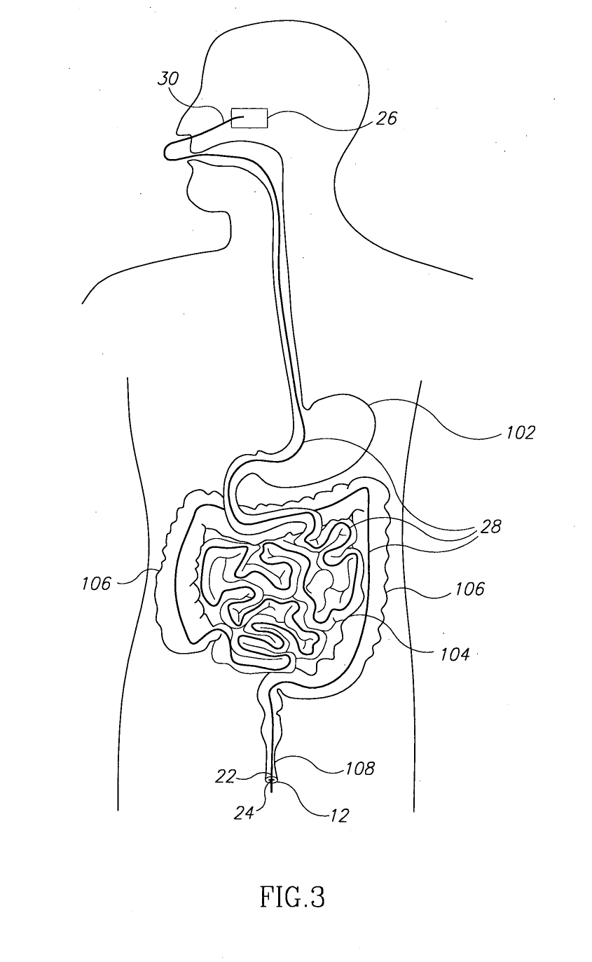

Gastrointestinal guidewire 28 has a guidewire body 34 extending from guidewire proximal end 30 to guidewire distal end 32. Guidewire body 34 is of a sufficient length to extend at least to the beginning of the colon of the gastrointestinal tract. According to some embodiments, the length of guidewire body 34 is sufficient to extend to a position within the colon, such as, for example, to the beginning of the rectum. Alternatively, guidewire body 34 may extend through an entire length of the gastrointestinal tract, such as, for example, to a position at or beyond the anus. For example, a guidewire having a length of approximately 22 meters may fit into a swallowable capsule if the diameter of the guidewire is sized accordingly (0.03 mm, for example). In the embodiments shown in FIGS. 1A, 1B and 2, initially, most of guidewire body 34 is positioned within interior portion 20 of introducing element 12. As introducing element 12 descends through the gastrointestinal tract in a direction depicted by arrow 42, progressively more of guidewire body 34 moves out of introducing element 12 and into the gastrointestinal tract via guidewire outlet 22 in a direction depicted by arrow 40, for example. In the embodiment shown in FIG. 1A, distal end 30 of gastrointestinal guidewire 28 remains attached to guidewire retainer 24 and proximal end of gastrointestinal guidewire 28 remains attached to anchoring element 26 during descent of introducing element 12. In this way, guidewire body 34 is able to be positioned within the gastrointestinal tract and held in place via anchoring element 26. In the embodiment shown in FIG. 1B, distal end 30 of gastrointestinal guidewire 28 remains attached to introducing element 12 at distal guidewire attachment point 38 and proximal end of gastrointestinal guidewire 28 remains attached to anchoring element 26 during descent of introducing element 12. However, in the embodiment shown in FIG. 1B, gastrointestinal guidewire 28 unwinds from inside out, rather than from outside in over a rotatable spool as depicted in FIG. 1A. In some embodiments, guidewire outlet 22 may further include a one-way valve which would allow gastrointestinal guidewire 28 to exit therefrom while preventing entry of fluids from the gastrointestinal tract into introducing element 12.

Introducing element 12 may advance through the entire gastrointestinal tract and be released through the rectum/anus. At this point gastrointestinal guidewire 28 is fully in place through the gastrointestinal tract and may be used as a scaffold for guiding a gastrointestinal device through the gastrointestinal system.

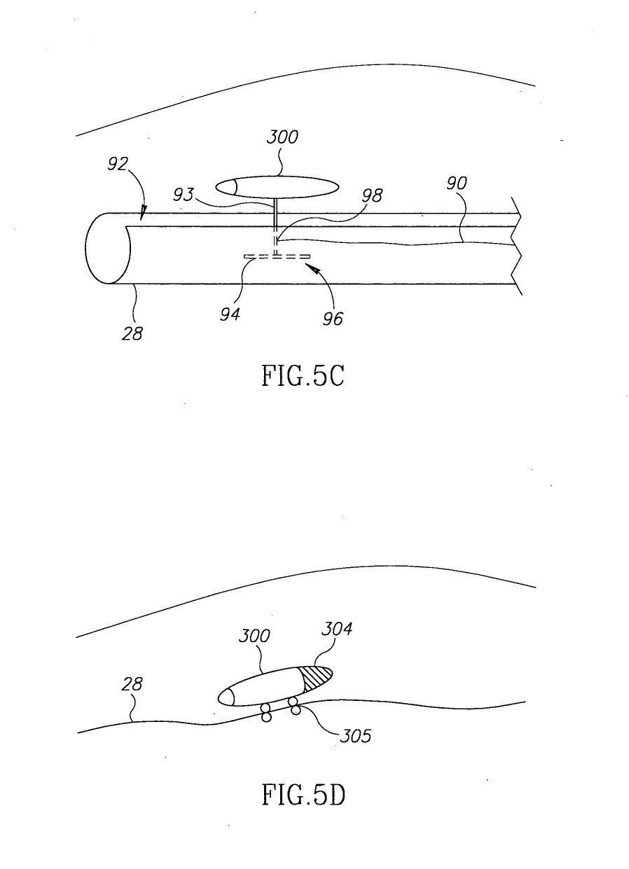

Gastrointestinal guidewire 28 may be made of any biocompatible or inert material, and should be of a material that is not likely to elicit an allergic or inflammatory response in the subject. Suitable materials include but are not limited to wire, elastic, string, cotton, wool, acrylic, nylon, plastic, polyester and combinations thereof. It is to be understood however, that the material must be of sufficient flexibility so as to enable gastrointestinal guidewire 28 to navigate around the tortuous path of the small and large intestines. Gastrointestinal guidewire 28 may further comprise a non-dissolvable coating, such as, for example, enamel, porcelain, a polymer (such as a polycarbon polymer, a polysaccharide polymer or a combination thereof), synthetic rubber or latex, or a combination thereof. The coating may be performed for example (and without limitation) by dipping, spraying or brush coating. In a particular embodiment, gastrointestinal guidewire comprises a line, as illustrated for example, in FIGS. 1A, 1B, and 2. In another particular embodiment, gastrointestinal guidewire 28 may be comprised of hollow tubing, as illustrated for example, in FIGS. 5A-5C. In some embodiments, gastrointestinal guidewire 28 is marked along its length with indicators that are detectable by an external detection method. Such indicators may include, for example, a periodic and/or nonperiodic pattern of features that may further assist in traction, such as teeth, holes, or grooves and may include, for example, a radiopaque material which is detectable by an external imaging technique. In some embodiments, gastrointestinal guidewire 28 is a wire comprised of a suitable plastic, metal or metal alloy, such as steel, nitinol, aluminum, titanium or combinations thereof. A diameter of gastrointestinal guidewire 28 may be in accordance with that of lines, cables, wires and similar instrumentation known for use in the art. Thus, for example, the diameter of the guidewire can be in a range of 0.01 mm to 10 mm, or in a range of 0.01 to 0.5 mm. In some embodiments, gastrointestinal guidewire 28 may be comprised of a biodegradable material.

Guidewire retainer 24 may be any suitable retainer for holding guidewire distal end 32 in interior portion 20 of introducing element 12 while allowing guidewire body 34 to be released from interior portion 20. For example, as shown in FIG. 1A, guidewire retainer may be a rotatable spool 36 around which gastrointestinal guidewire 28 is wound. In some embodiments, rotatable spool 36 may further comprise a pulley. Rotatable spool 36 may rotate clockwise or counter-clockwise. In some embodiments, rotatable spool 36 may further comprise an electric motor, which provides a force to drive rotatable spool 36. The force provided by the electric motor may be a torque force, a push force, a continuous force, an alternating intermittent force, and combinations thereof. Rotatable spool 36 may further include a locking mechanism, adapted to prevent further unspooling of gastrointestinal guidewire 28 once introducing element 12 has reached a desired point or is expelled through the anus.

In an alternative embodiment, as shown in FIG. 2, guidewire retainer 24 may be a solid or spring-like object, and gastrointestinal guidewire 28 may be positioned within interior portion 20 in any suitable configuration, while preventing wire entanglement, such as a zig-zag or other pattern.

Introducing element 12 is propelled by gastrointestinal peristalsis along the length of the gastrointestinal tract. The progressive movement of introducing element 12 allows for pulling and dragging of gastrointestinal guidewire 28 attached thereto and its release therefrom, until gastrointestinal guidewire 28 is eventually positioned along the length of the gastrointestinal tract.

Anchoring element 26 may be positioned in various locations. For example, anchoring element may be mounted outside the subject's mouth and in proximity to it, for example, on the face adjacent to or proximal to the mouth. In alternative embodiments, the mounting point may be within the oral cavity of the subject, such as on an inner cheek or a tooth, or adjacent to or proximal to the nasal cavity. In alternative embodiments, the mounting point may be anywhere on the upper torso of the subject, such as the neck, chest or back. The mounting point may also be inside the upper part of the gastrointestinal tract, e.g the stomach or the duodenum, after insertion of the introducing element via an endoscope.

Reference is now made to FIG. 3, which is an illustration of system 10 in place after introducing element 12 has emerged from the gastrointestinal tract. In the embodiment shown in FIG. 3, anchoring element 26 is placed on an outer surface of a cheek. Introducing element 12 moves through the entire gastrointestinal tract, including the stomach 102, small intestines 104 and large intestines 106, and exits via the anus 108. Gastrointestinal guidewire 28 is shown positioned through the entire gastrointestinal tract.

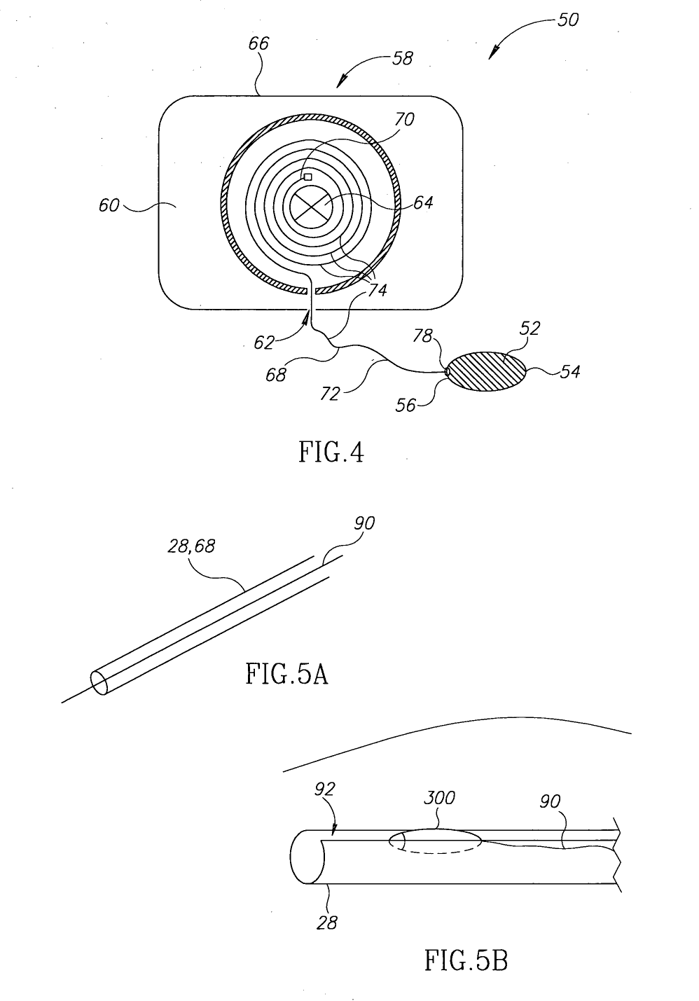

Reference is now made to FIG. 4, which is an illustration of a system 50 in accordance with alternative embodiments of the present invention. System 50 includes an introducing element 52 having an introducing element distal end 54 and an introducing element proximal end 56. Introducing element proximal end 56 is attached to a gastrointestinal guidewire 68 at a distal guidewire attachment point 78. System 50 further includes an anchoring element 66 having an exterior portion 58 and an interior portion 60. In some embodiments, interior portion 60 is at least partially hollow. A guidewire outlet 62 is positioned at one end of anchoring element 66. Guidewire outlet 62 is an opening of sufficient diameter to allow a guidewire to slide therethrough. Interior portion 60 of anchoring element 66 includes a guidewire retainer 64 for at least partially retaining a gastrointestinal guidewire 68 in interior portion 60, as will be explained hereinbelow.

Gastrointestinal guidewire 68 has a guidewire proximal end 70, a guidewire distal end 72 and a guidewire body 74 extending from guidewire proximal end 70 to guidewire distal end 72. Guidewire body 74 is of a sufficient length to extend through an entire length of the gastrointestinal tract. Initially, most of guidewire body 74 is positioned within interior portion 60 of anchoring element 66. As introducing element 52 descends through the gastrointestinal tract, it pulls gastrointestinal guidewire 68 along with it, causing progressively more of guidewire body 74 to move out of anchoring element 66 and into the gastrointestinal tract via guidewire outlet 62. Guidewire proximal end 70 remains attached to guidewire retainer 64 and guidewire distal end 72 remains attached to introducing element 52 during descent of introducing element 52. In this way, guidewire body 74 is able to be positioned within the gastrointestinal tract and held in place via anchoring element 66. Introducing element 52 may descend through the entire gastrointestinal tract and may be released at the anus. At this point gastrointestinal guidewire 68 is fully in place through the gastrointestinal tract and may be used as a scaffold for guiding a gastrointestinal device through the gastrointestinal system.

In one embodiment, gastrointestinal guidewire 28, 68 is a hollow tube, as shown in FIG. 5A. An additional wire 90 may be placed through the hollow tube. In some embodiments, as shown in FIG. 5B, the hollow tube may include a slit 92, and wire 90 may be used as a scaffold for a gastrointestinal device 300. Gastrointestinal device 300 may be, for example, a diagnostic or therapeutic device, such as, for example, comprises one or more of a capsule, an imaging device (such as a still and/or video camera and/or an illumination unit), an endoscope, a biopsy device, or a luminal content sampling device, electrocautery device, drug delivery device, surgical device or instrument, or a combination thereof. According to some embodiments, an imaging device is contained in or within a capsule. According to some embodiments an angle of gastrointestinal device 300 is adjustable with regard to a longitudinal axis of the gastrointestinal tract.

In one embodiment, as shown in FIG. 5C, gastrointestinal device 300 includes a protrusion 94 for inserting into slit 92. Protrusion 94 may be, for example, T-shaped and may include a horizontal piece 96 and a vertical piece 98. Horizontal piece 96 may be positioned within slit 92, and vertical piece 98 may be attached to an image detection means 300 such as a camera, located outside and adjacent to the tubing. Protrusion 94 may be further attached to a control wire 90 wherein manipulation of control wire 90 enables forward and backward movement of protrusion 94, causing the image detection means to slide over the hollow tube in the desired direction. A gastrointestinal device 300 which may be used in conjunction with the guidewire will typically be an autonomous imaging system, including for example, an imaging system an optical system, an imaging camera (such as a CMOS camera, a CCD camera, or another suitable imaging device) and an illumination unit including one or more of light sources. Such an imaging system is described for example, in U.S. Pat. No. 5,604,531 and International Publication Number WO 01/65995. According to some embodiments, gastrointestinal device 300 may be manipulated to change the image-capturing angle (i.e. the angle of the camera, or the angle of the entire capsule) in relation to the longitudinal axis of the lumen of the gut, so as to optimize maximal visual data capturing.

Alternatively, gastrointestinal device 300 may be any gastrointestinal device known and used by those of skill in the art, including wires, endoscopes, diagnostic or therapeutic devices, tubes and combinations thereof. As many of these devices comprise hollow channels along their shafts, they can be manually manipulated to slide over the guidewire 28 inserted through their hollow channels, as is known to those skilled in the art of introducing medical tubings into body organs over pre-placed guide-wires.

According to some embodiments, gastrointestinal device is self-propelled along the wire by tension in guidewire 28 caused by anchoring each of the ends of guidewire 28 to anchoring points. For example, guidewire 28 proximal end may be anchored via anchoring element 26 the face of the subject, guidewire 28 distal end may be anchored to a region around the anus of the subject. Alternatively, a mechanism for creating local tension on guidewire 28 may be provided by two arms extending from the capsule and gripping the wire on the forward and backward sides of the capsule.

In some embodiments, gastrointestinal device 300 may be comprised of a magnetable substance which can be used to navigate the movement of the device along the line, by application of a second magnet externally over the subject's body operated by a practitioner.

In some embodiments, gastrointestinal device 300 may include a motor and a mechanism for self-propulsion along gastrointestinal guidewire 28, as shown in FIG. 5D. In this embodiment, gastrointestinal device 300 comprises a motor unit 304 and wheels 305. The wheels 305 may be included in a mechanism which is external to the gastrointestinal device 300, as in FIG. 5D. In an alternative embodiment, wheels 305 may be contained within the interior of device 300, wherein guidewire 28 enters and exits device 300 through outlets at its two ends, and is in contact with wheels 305 inside the gastrointestinal device 300. Wheels 305 may include double wheels and may be attached to guidewire 28 between the double wheels to preclude slippage off the track. Wheels 305 may be propelled by motor 304, causing the wheels to spin along guidewire 28 and propel gastrointestinal device 300 along guidewire 28 in the gastrointestinal tract, using guidewire 28 as a track line. The motor unit 304 can be controlled by an external operator through various signals to determine speed and direction of device movement. The gastrointestinal guidewire 28 may have a configuration other than a line, for example, as a track, flexible rail, chain, slide or belt, and may have periodic indentations to facilitate the grasp of the wheels mechanism, which may assume a tooth-wheel like configuration. The spooled wire, or the tubes or wires subsequently introduced along it, can be comprised of a material able to conduct electricity, to supply electricity to the motor.

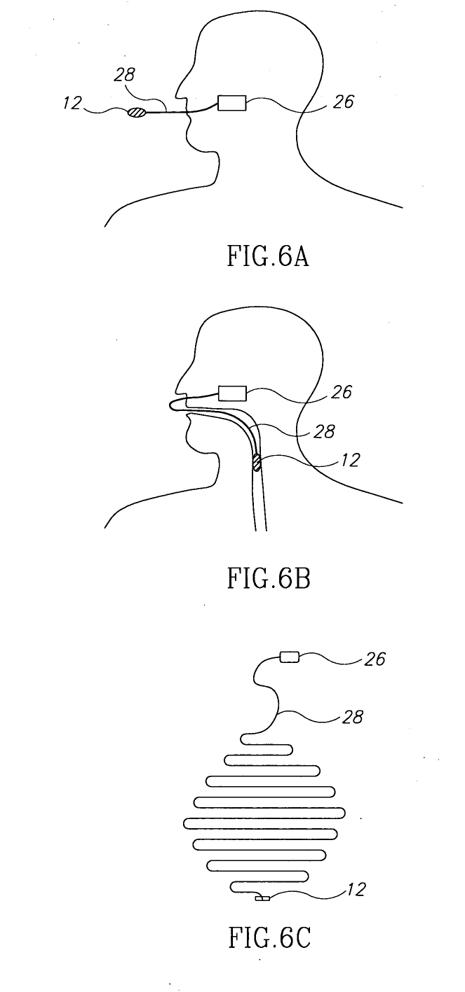

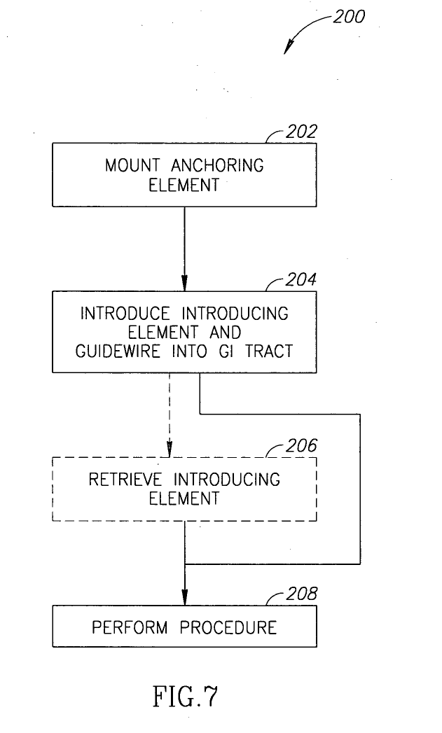

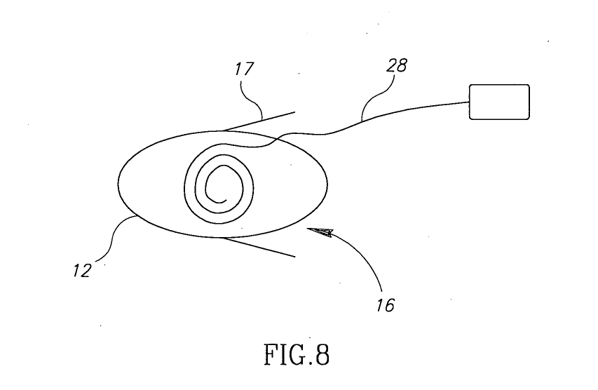

Reference is now made to FIGS. 6A-6C, taken together with FIG. 7, which are schematic illustrations and a flow-chart illustration, respectively, of a method 200 for controlled advancement of a gastrointestinal device, in accordance with embodiments of the present invention.

First, anchoring element 26 is mounted (step 202) onto the body or a location outside of the body of the subject, as shown in FIG. 6A. The subject is most often a human, but may be any other mammal. Mounting may be accomplished with suitable adhesives including adhesive tapes, as are known to one of ordinary skill in the art. Other means include, but are not limited to glue, tape, gel, suction cups, close-fitting nylon wrap, bands, molds and combinations thereof. Alternately, or in addition, the mounting may be accomplished using grip fasteners attached to anchoring element 26 which fasten around the head of the subject for keeping the device in the desired position. Sutures or staples may also be used, but are less desirable because of possible pain involved in their placement or removal. The mounting point on the body of the subject may be external to the gastrointestinal tract, and may be, for example, on the face adjacent to or proximal to the mouth. In alternative embodiments, the mounting point may be within the oral cavity of the subject, or adjacent to or proximal to the nasal cavity. In alternative embodiments, the mounting point may be anywhere on the upper torso of the subject, such as the neck, chest or back. In alternative embodiments the mounting point may be in the stomach or duodenum of the subject, which can be mounted via an endoscopy procedure.

Next, introducing element 12 is introduced (step 204) into the gastrointestinal tract, as shown in FIG. 6B, and introducing element 12 is propelled through the entire length of the gastrointestinal tract, thus positioning gastrointestinal guidewire 28 within the gastrointestinal tract (not shown), as shown in a highly schematic manner in FIG. 6C, which shows that guidewire 28 is flexibly bent along the contours of the gastrointestinal tract. In one embodiment, the introducing step comprises oral administration of introducing element 12 to the subject, such as, by example, swallowing. The introducing step may however, comprise manually manipulating the guidewire 28 through the nasal nares into the oral cavity, pulling it outside the mouth to secure the introducing element 12 to it, and then introducing the introducing element 12 with guidewire 28 attached thereto into the esophagus and/or stomach and/or the duodenum of the subject by swallowing or other means. This allows the guidewire to extend from the gastrointestinal tract to the anchoring point outside the body through the nasal nares rather than the mouth, so as to reduce discomfort to the subject. Alternatively, the introducing may be accomplished using an endoscope to position introducing element 12 into the stomach or the duodenum of the subject. Mounting of anchoring device to the internal surface of the stomach wall can then be accomplished by various methods known to those skilled in the art, such as, but not limited to, endoscopic suturing or clipping.

Once introducing element 12 is introduced into the gastrointestinal tract of the subject, the method of the invention relies, at least partially, upon gastrointestinal peristalsis for movement of introducing element 12 through the GI tract and subsequent positioning of gastrointestinal guidewire 28 within the GI tract. The gastrointestinal peristalsis may be unassisted, or may be augmented by administration of pharmaceutical agents which act as motility enhancers, in order to increase the frequency and/or strength of the peristaltic contractions in the subject. The administration of such agents may be prior to the initiation of the method of the invention, and/or during the course of disbursing the guidewire. The selection and dose of a suitable agent or agents may be readily determined by one of ordinary skill in the art. In some embodiments, an augmenting force may be required. The force required may be provided by an electric motor associated with system 10. A suitable electric motor may be selected to provide one or more types of mechanical forces to enhance movement of weighting mechanism in the gastrointestinal tract. A suitable mechanical force may be selected from a torque force, a push force, a continuous force, an intermittent force, and combinations thereof.

The pulling force of introducing element 12 as it moves through the gastrointestinal tract functions to drag the attached guidewire and to progressively release gastrointestinal guidewire 28 from guidewire retainer 24, such as, by example, unspooling gastrointestinal guidewire 28 from spool 36. In some embodiments, system 10 may comprise an electric motor which provides a force to release gastrointestinal guidewire 28. The progress of the release of gastrointestinal guidewire 28 may be monitored, for example, by an external detection method, wherein introducing element 12 and/or gastrointestinal guidewire 28 comprise indicators that are detectable by an external detection method.

Introducing element 12 is then optionally retrieved (step 206) at the distal end of the gastrointestinal tract once it passes through the anus, as shown with dashed lines in FIG. 7. Retrieval of introducing element 12 may be carried out following its extrusion through the anus of the subject, or alternately, using an endoscope to manipulate introducing element 12 out through the anus while still attached to gastrointestinal guidewire 28. To complete the retrieval step, introducing element 12 may be detached from gastrointestinal guidewire 28 by means of a dedicated fastening means, such as a clip, pin or other fastening means. It is to be understood that upon recovery of the weighting apparatus, gastrointestinal guidewire 28 remains positioned throughout the length of the gastrointestinal tract. Alternatively, introducing element 12 may be degraded within the body of the subject.

Once gastrointestinal guidewire 28 is in place, a diagnostic or therapeutic procedure may be performed (step 208) by using gastrointestinal guidewire 28 as a scaffold for a diagnostic or therapeutic device. It should be readily apparent that the amount of time between the positioning of gastrointestinal guidewire 28 and the beginning of the diagnostic or therapeutic procedure may vary. Thus, the time it takes for introducing element 12 to descend through the gastrointestinal tract, which may vary from individual to individual, is of little significance, since the procedure takes place some time afterwards. It may take place immediately afterwards, or hours or days afterwards. Diagnostic gastrointestinal procedures include gastroduodenoscopy of the upper gastrointestinal tract, colonoscopy of the large intestine, enteroscopy of the small intestine, pan-endoscopy of the entire gastrointestinal tract, capsule endoscopy of at least a portion of the gastrointestinal tract, including capsule studies to obtain visual imaging of the gastrointestinal tract or for assessment of various parameters of at least a portion of the gastrointestinal tract. Such parameters include pH, temperature, pressure, and concentration of various biochemical analytes and identifiers. Further diagnostic and therapeutic procedures may comprise biopsy of at least a portion of the gastrointestinal tract, sampling at least a portion of the luminal contents of at least a portion of the gastrointestinal tract, delivery of a therapeutic modality (such as drug delivery or electrocauterization or a combination thereof) to at least a portion of the gastrointestinal tract, performing a surgical procedure on at least a portion of the gastrointestinal tract or cleaning of at least a portion of the gastrointestinal tract. Diagnostic and therapeutic gastrointestinal procedures can comprise insertion of various instruments into the gastrointestinal tract, including graspers, blades, clamps, tissue collecting baskets, scalpels, stents, catheters, suturing devices, forceps, dilatation balloons, injectors, forceps, anchors, drug applicators, samplers, biopsy samplers, electrodes, suction tubes, temperature sensors, optical sensors, electrical impedance sensors, pH meters, electrocautery devices, argon plasma applicators and others. Furthermore, diagnostic and therapeutic gastrointestinal procedures can include various manipulations and functions, including tissue cutting, tissue welding, suturing, cauterizing, ablating, clamping, biopsy and tissue sampling, optical sensing, chemical sensing, application of a substance or substances, injection of a substance or substances, imaging, and temperature sensing.

Gastrointestinal device 300 may be externally manipulated (ie, manipulated from outside the body) in one of several ways. For example, in some embodiments, gastrointestinal device 300 includes wheels which can be controlled remotely, and made to spin in opposite directions or locked in place. Alternatively, a second wire may be attached to the gastrointestinal device 300 and may be used to push or pull it over gastrointestinal guidewire 28. Thus, gastrointestinal device 300 may be manipulated, which allows for it to be held in place at a particular location, pulled back to view or sample an earlier portion of the gastrointestinal tract, pushed along so that it reaches the large intestines while the battery is still operable, or any other suitable manipulations.

Gastrointestinal device 300 may be guided through the gastrointestinal tract by at least one of speeding up, slowing down, reversal and stopping of gastrointestinal device 300, or changing an angle of gastrointestinal device 300 with regard to the longitudinal axis of the gastrointestinal tract.

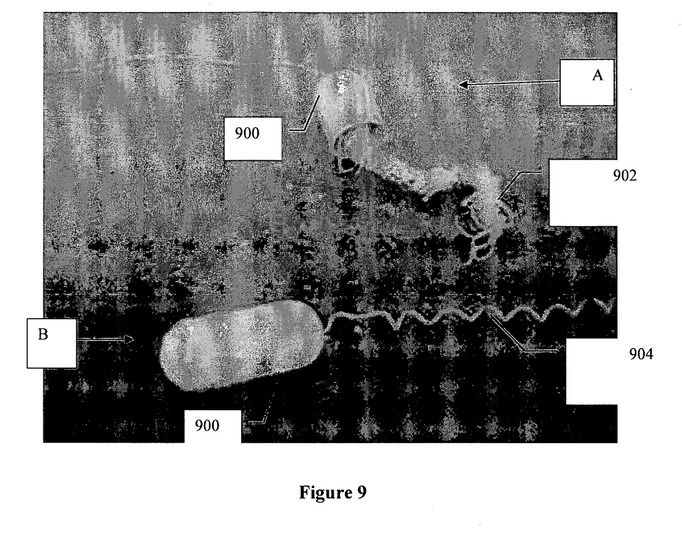

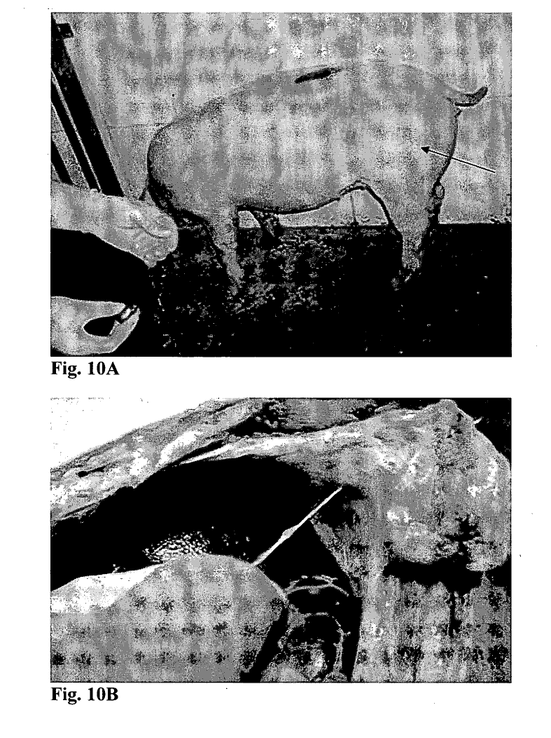

FIG. 9 shows two views of an exemplary introducing element and guidewire according to the present invention. In a top view, labeled with an arrow and the letter “A”, an introducing element 900 is shown open; the coiled guidewire 902 is shown emerging from the introducing element 900. When the introducing element 900 is closed, the guidewire 902 would be contained within the introducing element 900 and would not be visible, except for a portion 904 emerging from an outlet (not shown).

In a bottom view, labeled with an arrow and the letter “B”, introducing element 900 is shown closed, with the majority of the guidewire 902 contained within and the portion 904 emerging from the outlet (not shown). In this exemplary embodiment, introducing element 900 is shown as a capsule for the purpose of illustration only and without any intention of being limiting in any way.

### EXAMPLES

Reference is now made to the following examples, which together with the above description illustrate the invention in a non limiting fashion.

### Example 1

### In Vitro Testing on Pig Intestinal Section

A wire of diameter 0.034 cm was used to determine maximal force needed for a guidewire to cause a tear of the intestinal wall. The intestinal section was cut in its longtitudinal axis, laid open on a rigid surface, and fixed. The wire was then manually pressed and pulled over the luminal (mucosal) side of the intestinal section several times, with gradual increase of manual pressure applied until maximal force was exerted. No macroscopic tear of the intestinal wall was observed.

### Example 2

### In Vivo Testing in Pig

22 m of wire of 0.034 cm diameter were rolled and inserted into an oval shaped introducing element measuring 30×11 mm. The wire was composed of polyamide and the introducing element was composed of polycarbonate. The wire was rolled upon itself such that pulling on its proximal end resulted in its unwinding in an inside-out fashion. The proximal end was fixed to the inside wall of the introducing element. The introducing element was provided with an outlet on one side of its longitudinal axis, through which 30 cm of wire were threaded and left outside of the introducing element.

A 60 kg female pig was studied, after 24 hours food fast (water ad libitum).

The pig was anesthetized as follows:

Pre-medication: Ketamine HCl 10 mg/kg+Xylazine 1-2 mg/kg IM

After_vain canulation diazepam 10 mg/pig-IV.

Induction: Isoflorane 5% mask
Maintenance: After intubation, Isoflorane 1-3% delivered via positive pressure ventilation (IPPV) utilizing 100% oxygene (2 L/min).
The animal received Ampicillin HCl 1 gr IV 15 minutes prior to the procedure.

Gastroscopy was performed and abdominal trans-illumination marked. A gastroscope was re-introduced with a Roth-net grasping the oval-shaped introducing element. Percutaneous puncture of stomach was performed with a 13 g hollow needle, and an alligator biopter inserted through the needle. The free proximal end of the introducing element wire was grasped by the alligator biopter and the wire pulled through the needle. The wire was knotted to the sub-cutaneous abdominal tissue adjacent to the anterior aspect of the gastric body. The introducing element was freed from the Roth-net into the lumen of the stomach, and the endoscope withdrawn; however, the introducing element and wire were clearly visible in the lumen of the stomach through the endoscope prior to withdrawal (picture not shown). Extubation was performed after anesthesia cessation. The pig was sent back to the animal facility and maintained on a normal diet for 3 days, with close observation. Normal behavior and eating patterns were observed.

On the third day following the procedure, the introducing element with attached wire was expelled from anus of the pig during a normal bowel movement. The wire was cut from the anus and maintained with the capsule in a container. The pig was sent for laparotomy (euthanasia and necropsy) at Sheba Hospital, Israel.

The appearance of the pig was normal, except for a somewhat distended abdomen. The body temperature of the pig was normal (39.2° C.).

Following euthanasia by Pental 5 cc+KCL 15% 20 ml, the abdomen was opened. A small amount of free peritoneal fluid in normal quantity was observed, with somewhat turbid appearance. This was aseptically collected and sent for culture for anaerobic and aerobic bacteria.

The wire was found to be still attached to the abdominal wall, coursing to the stomach wall through 2 liver lobes (resulting from the percutaneous needle puncture during the gastrostomy). No frank hemorrhage was noted. The mesentery appeared mildly congested but no intestinal perforation was seen. The wire was observed entering the anterior gastric wall, with omental adhesion noted around the insertion point.

After separation of the omentum, the stomach was sectioned at the pylorus, and the wire observed coursing from the stomach to the duodenum. The stomach was then resected opened along the lesser curvature, and its contents spilled and washed with fresh tap water. The wire was observed being attached to the gastric body and coursing towards the pylorus. The mucosa appeared normal.

Subsequently, the small intestine was resected at the ileo-cecal valve and the wire was observed to be coursing from the ileum to the cecum.

The small bowel was then resected open along its longtitudinal axis at 10 cm intervals, and the wire observed to be coursing along its entire length and with normal-appearing mucosa. The large bowel was also sectioned with similar results observed.

Four intestinal specimens were taken from intestine, immersed in paraformaldehye 4% and sent to the pathology laboratory. The pig corpse was sent for burial. Peritoneal fluid culture was received negative for aerobic and anaerobic bacteria.

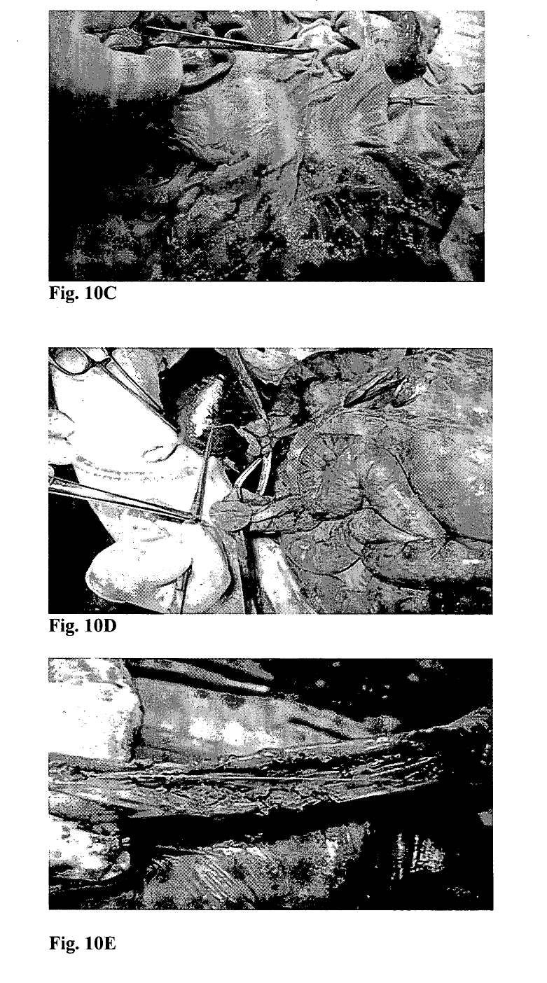

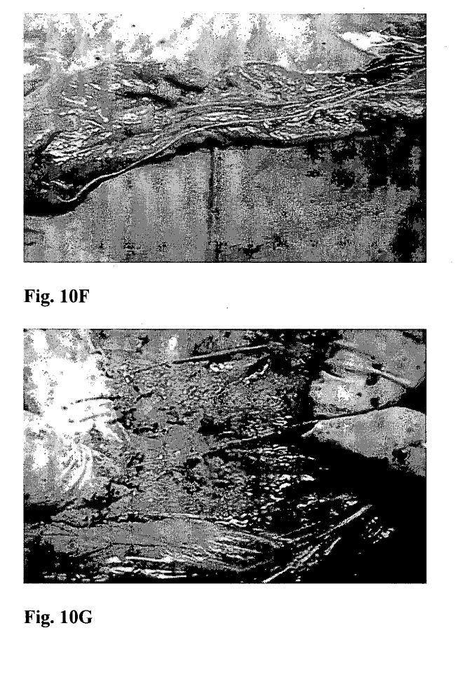

FIG. 10A shows the wire extending from the anus of the pig (arrow) and being held by the hand of the investigator, along with the expelled introducing element. FIG. 10B shows the attachment of the wire to the abdominal wall adjacent to anterior wall of gastric body. The anchored wire extends along the stomach (FIG. 100), then passes from the stomach to the duodenum (FIG. 10D), along the small bowel, from the ileum to the cecum (FIGS. 10E and 10F), and along the large bowel (FIG. 10G).

While certain features of the present invention have been illustrated and described herein, many modifications, substitutions, changes, and equivalents may occur to those of ordinary skill in the art. It is, therefore, to be understood that the appended claims are intended to cover all such modifications and changes as fall within the true spirit of the present invention.

1. A system for introduction of a gastrointestinal device through an oral cavity of a subject to a gastrointestinal tract of the subject, the system comprising:
a) an introducing element having an introducing element distal end, an introducing element proximal end, an exterior portion and an interior portion, said introducing element being adapted for introduction through said oral cavity and for being swallowed by the subject;
b) a guidewire attachment point at said introducing element; and
c) an anchoring element external to said introducing element for anchoring to the subject; and
d) a gastrointestinal guidewire having a guidewire proximal end, a guidewire distal end, and a guidewire body extending from said guidewire proximal end to said guidewire distal end, said guidewire body having a length sufficient to be positioned from said oral cavity through at least to the beginning of the colon of the gastrointestinal tract, said guidewire proximal end attached to said anchoring element, said guidewire distal end positioned within said interior portion and attached to said guidewire attachment point, and at least a portion of said guidewire body configured to be released from said interior portion into the gastrointestinal tract as said introducing element traverses at least a portion of the gastrointestinal tract, said gastrointestinal guidewire configured to be used as a scaffold for guiding a gastrointestinal device through the gastrointestinal tract.

2. The system of claim 1, wherein said length of said guidewire body is sufficient to be positioned to a position within the gastrointestinal tract chosen from the group consisting of within the colon, at the beginning of the rectum, at or beyond the anus, through the entire length of the gastrointestinal tract.

3-9. (canceled)

10. The system of claim 1, wherein said introducing element comprises a material selected from the group consisting of a digestible material, gelatin, a dissolvable material, a biodegradable material, an indigestible material, non-dissolvable material, and a combination thereof.

11-16. (canceled)

17. The system of claim 1, wherein said introducing element comprises a detection means chosen from the group consisting of: a detection means, a sensor, an image detection means, an externally detectable indicator, radiopaque markers, RFID tag, metal component detectable by a magnetometer, pH sensor, pressure sensor, luminal diameter sensor, peristaltic contraction sensor, and a combination thereof.

18-20. (canceled)

21. The system of claim 1, wherein said gastrointestinal guidewire comprises at least one material selected from the group consisting of: an electrically conductive material, a biodegradable material, a non-biodegradable material, a radiopaque material, a plastic, a metal, metal alloy, steel, nitinol, aluminium, titanium, a hollow tube, a hollow tube comprising a slit, or a combination thereof.

22-33. (canceled)

34. The system of claim 1 wherein said guidewire attachment point is configured to allow detachment of said guidewire from said introducing element.

35-39. (canceled)

40. The system of claim 1, wherein a diameter of said guidewire is in the range of from about 0.01 mm to about 10 mm.

41. (canceled)

42. The system of claim 1, wherein the gastrointestinal device further comprises a diagnostic or therapeutic device modified to be externally manipulated along said gastrointestinal guidewire, said diagnostic or therapeutic device chosen from the group consisting of capsule, an imaging device, an endoscope, a biopsy device, a luminal content sampling device, electrocautery device, drug delivery device, surgical device or instrument, a still camera, a video camera, an illumination unit, a camera and an illumination unit, magnet, magnetable material, an externally controllable wire, and an externally controllable motor, or a combination thereof.

43-45. (canceled)

46. The system of claim 42, wherein an angle of said gastrointestinal device is adjustable with regard to longitudinal axis of said gastrointestinal tract.

47-53. (canceled)

54. A method for controlling a gastrointestinal device within the gastrointestinal tract of an individual, the method comprising:
providing an introducing element configured to progress through the gastrointestinal tract, an anchoring element, and a guidewire attached to said introducing element and said anchoring element;
anchoring said anchoring element to a location external to or in a proximal part of the gastrointestinal tract;
introducing said introducing element into the gastrointestinal tract and allowing said introducing element to traverse at least a portion of the gastrointestinal tract by peristaltic contractions, wherein during said traversal, said guidewire is positioned through the traversed portion of the gastrointestinal tract; and
guiding a gastrointestinal device through the gastrointestinal tract via said guidewire, wherein said guiding includes externally controlling at least one movement of said gastrointestinal device.

55. The method of claim 54, wherein said guidewire is initially at least partially positioned within said introducing element, and wherein during said traversal by said introducing element, said guidewire is configured to be released from said introducing element.

56. The method of claim 54, wherein said anchoring is done by attaching said anchoring element to a location selected from the group consisting of an oral cavity, a nasal cavity, a stomach, a duodenal cavity, an upper torso, and a location outside of a body of the individual.

57. The method of claim 54, wherein said introducing is done by swallowing.

58. The method of claim 54, wherein said allowing said introducing element to traverse at least a portion of the gastrointestinal tract comprises allowing said introducing element to traverse an entire gastrointestinal tract.

59. The method of claim 54, wherein said guiding a gastrointestinal device comprises at least one of: providing a remotely controllable motor to said gastrointestinal device and externally controlling movement of said gastrointestinal device via same remotely controllable motor or providing a controlling wire to said gastrointestinal device and externally controlling movement of said gastrointestinal device via said controlling wire, or application of an external magnet to navigate a magnetable gastrointestinal device over the guidewire.

60. The method of claim 54, wherein said guiding comprises at least one of: speeding up, slowing down, reversal and stopping of the gastrointestinal device, or changing an angle of said gastrointestinal device is with regard to longitudinal axis of said gastrointestinal tract.

61. The method of claim 54, further comprising removing said introducing element from the gastrointestinal tract.

62. The method of claim 61, wherein said removing comprises removing through, or expulsion from, an anus.

63. The method of claim 61, wherein said removing comprises degradation of said introducing element.

64. The method of claim 54, further comprising removing said introducing element from the gastrointestinal tract while leaving said guidewire positioned in said traversed portion of the gastrointestinal tract.

65. The method of claim 54, wherein said introducing is done by insertion of an endoscope.

66. The method of claim 61, wherein said removing is done by use of an endoscope.

67-73. (canceled)

74. The system of claim 1, wherein said introducing element is characterized according to at least one or more configuration chosen from the group consisting of:
a. said introducing element distal end is lighter than said introducing element proximal end;
b. said introducing element distal end is narrower than said introducing element proximal end;
c. said introducing element having a spherical shape;
d. said introducing element having a hemispherical shape;
e. said introducing element having a tapered shape;
f. said introducing element having a conical shape;
g. said introducing element having a cylindrical shape;
h. said introducing element having an ovoid shape;
i. said introducing element proximal end further comprises an expandable portion;
j. said introducing element proximal end further comprises an expandable portion comprising at least two outwardly extendable wings;
k. said introducing element proximal end further comprises an expandable portion expandable portion adept to expand following traversal.

75. The system of claim 1, wherein said introducing element comprises a guidewire retainer for retaining at least a portion of said guidewire in said introducing element having configuration chosen from the group consisting of:
a. said interior portion guidewire retainer comprises a rotatable spool;
b. wherein said guidewire retainer comprises a rotatable spool comprising means chosen from the group consisting of a pulley, an electric motor and a locking mechanism;
c. wherein said interior portion guidewire retainer comprises a spring-like object;
d. or any combination thereof.

76. The system of claim 1, wherein said introducing element is characterized according to at least one or more configuration chosen from the group consisting of:
a. said introducing element proximal end further comprises a guidewire outlet;
b. said introducing element proximal end further comprises a guidewire outlet comprising a one way valve;
c. said introducing element comprises said guidewire attachment point at said introducing element interior wall;
d. said introducing element comprising said guidewire attachment point at said exterior wall;
e. said introducing element comprises said guidewire attachment point at said introducing element at an interior wall or an exterior wall of said introducing element, and a guidewire outlet at said introducing element proximal end;
f. said introducing element comprising said guidewire attachment point at said introducing element at an interior wall or an exterior wall of said introducing element, and a guidewire outlet at said introducing element proximal end, wherein said guidewire outlet comprises a one way valve;
g. any combination thereof.

77. The system of claim 1, wherein said gastrointestinal guidewire configured to be used as a scaffold for guiding a gastrointestinal device or therapeutic device through the gastrointestinal tract is used to guide a therapeutic device or gastrointestinal device chosen from the group consisting of a capsule, an imaging device, an endoscope, a biopsy device, a luminal content sampling device, electrocautery device, drug delivery device, surgical device or instrument, a still camera, a video camera, an illumination unit, a camera and an illumination unit, magnet, magnetable material, an externally controllable wire, an externally controllable motor, or a combination thereof.

78. A system for guidance introduction of a gastrointestinal device through an oral cavity of a subject to a gastrointestinal tract of the subject, the system comprising:
a) an introducing element having an introducing element distal end, an introducing element proximal end, an exterior portion and an interior portion, said introducing element being adapted for introduction into a portion of the gastrointestinal tract with an endoscope; and
b) a guidewire attachment point at said introducing element; and
c). an anchoring element external to said introducing element for anchoring to the subject; and
d) a gastrointestinal guidewire having a guidewire proximal end, a guidewire distal end, and a guidewire body extending from said guidewire proximal end to said guidewire distal end, said guidewire body having a length sufficient to be positioned from said oral cavity through at least to the beginning of the colon of the gastrointestinal tract, said guidewire proximal end attached to said anchoring element, said guidewire distal end positioned within said interior portion and attached to said guidewire attachment point, and at least a portion of said guidewire body configured to be released from said interior portion into the gastrointestinal tract as said introducing element traverses at least a portion of the gastrointestinal tract, said gastrointestinal guidewire configured to be used as a scaffold for guiding a gastrointestinal device through the gastrointestinal tract.

79. A system for introduction of a gastrointestinal device through an oral cavity of a subject to a gastrointestinal tract of the subject, the system comprising:
a) an introducing element having an introducing element distal end, an introducing element proximal end, an exterior portion and an interior portion, said introducing element being adapted for introduction through said oral cavity and for being swallowed by the subject, wherein said introducing element is in the form of a swallowable capsule; and
b) a guidewire attachment point at said introducing element; and
c) an anchoring element external to said introducing element for anchoring to the subject; and
d) a gastrointestinal guidewire having a guidewire proximal end, a guidewire distal end, and a guidewire body extending from said guidewire proximal end to said guidewire distal end, said guidewire body having a length sufficient to be positioned from said oral cavity through at least to the beginning of the colon of the gastrointestinal tract, said guidewire proximal end attached to said anchoring element, said guidewire distal end positioned within said interior portion and attached to said guidewire attachment point, and at least a portion of said guidewire body configured to be released from said interior portion into the gastrointestinal tract as said introducing element traverses at least a portion of the gastrointestinal tract, said gastrointestinal guidewire configured to be used as a scaffold for guiding a gastrointestinal device through the gastrointestinal tract.

80. The system of claim 79, wherein said introducing element is characterized according to at least one or more configuration chosen from the group consisting of:
a) said introducing element distal end is lighter than said introducing element proximal end;
b) aid introducing element distal end is narrower than said introducing element proximal end;
c) said introducing element comprises a material selected from the group consisting of a digestible material, gelatin, a dissolvable material and a biodegradable material, an indigestible material, non-dissolvable material, and a combination thereof;
d) said introducing element comprises a guidewire retainer for retaining at least a portion of said guidewire in said introducing element wherein said interior portion guidewire retainer comprises a rotatable spool;
e) said introducing element comprises a guidewire retainer for retaining at least a portion of said guidewire in said introducing element wherein said guidewire retainer comprises a rotatable spool comprises means chosen from the group consisting of a pulley, an electric motor and a locking mechanism;
f) said introducing element comprises a guidewire retainer for retaining at least a portion of said guidewire in said introducing element wherein said interior portion guidewire retainer comprises a spring-like object; and
g) any combination thereof.

81. The system of claim 79 wherein said introducing element comprises a detection means selected from the group consisting of: a detection means, a sensor, an image detection means, an externally detectable indicator, radiopaque markers, RFID tag, metal component detectable by a magnetometer, pH sensor, pressure sensor, luminal diameter sensor, peristaltic contraction sensor, and a combination thereof.

82. The system of claim 79, wherein the gastrointestinal device further comprises a diagnostic or therapeutic device modified to be externally manipulated along said gastrointestinal guidewire, said diagnostic or therapeutic device chosen from the group consisting of capsule, an imaging device, an endoscope, a biopsy device, a luminal content sampling device, electrocautery device, drug delivery device, surgical device or instrument, a still camera, a video camera, an illumination unit, a camera and an illumination unit, magnet, magnetable material, an externally controllable wire, and an externally controllable motor, or a combination thereof.

83. The system of claim 79, wherein the gastrointestinal device further comprises a diagnostic or therapeutic device modified to be externally manipulated along said gastrointestinal guidewire, said diagnostic or therapeutic device chosen from the group consisting of capsule, an imaging device, an endoscope, a biopsy device, a luminal content sampling device, electrocautery device, drug delivery device, surgical device or instrument, a still camera, a video camera, an illumination unit, a camera and an illumination unit, magnet, magnetable material, an externally controllable wire, and an externally controllable motor, or a combination thereof.

84. The system of claim 83, wherein an angle of said gastrointestinal device is adjustable with regard to longitudinal axis of said gastrointestinal tract.

85. The system of claim 79, wherein said gastrointestinal guidewire configured to be used as a scaffold for guiding a gastrointestinal device or therapeutic device through the gastrointestinal tract is used to guide a therapeutic device or gastrointestinal device chosen from the group consisting of a capsule, an imaging device, an endoscope, a biopsy device, a luminal content sampling device, electrocautery device, drug delivery device, surgical device or instrument, a still camera, a video camera, an illumination unit, a camera and an illumination unit, magnet, magnetable material, an externally controllable wire, an externally controllable motor, or a combination thereof.
